(19) United States
(12) Patent Application Publication
(10) Pub. No.: US 2025/0179056 A1
(43) Pub. Date: Jun. 5, 2025

(54) NOVEL SULFATE SALT FORMS OF ISOCHROMAN-IMIDAZOLE STRUCTURED ALPHA-2A ADRENOCEPTOR AGONIST

(71) Applicant: ORION CORPORATION, Espoo (FI)

(72) Inventors: Anna HAAPALINNA, Espoo (FI); Teemu HEIKKILÄ, Espoo (FI); Mihaela POP, Amsterdam (NL)

(21) Appl. No.: 18/832,210

(22) PCT Filed: Jan. 23, 2023
(86) PCT No.: PCT/FI2023/050047

§ 371 Date: Jul. 23, 2024

(30) Foreign Application Priority Data
Jan. 24, 2022 (FI) 20225050

(51) Int. Cl. C07D 405/04; A61K 31/4178

(52) U.S. Cl. CPC C07D 405/04; A61K 31/4178

(57) ABSTRACT

The present disclosure relates to novel sulfate salt forms of 2-(5-methoxyisochroman-1-yl)-4,5-dihydro-1H-imidazole (I). Compound (I) is a selective alpha2A adrenoceptor agonist and is useful in the treatment of anxiety, and for use as a sedative or analgesic agent, and other diseases were alpha2A agonism is desired.

### TECHNICAL FIELD

The present disclosure relates to novel sulfate salt forms of 2-(5-methoxyisochroman-1-yl)-4,5-dihydro-1H-imidazole (I) and to preparation thereof. Furthermore, the disclosure relates to pharmaceutical composition comprising at least one sulfate salt form of 2-(5-methoxyisochroman-1-yl)-4,5-dihydro-1H-imidazole (I).

### BACKGROUND OF THE INVENTION

Tasipimidine is the International Nonproprietary Name (INN) of the compound 2-(5-methoxyisochroman-1-yl)-4,5-dihydro-1H-imidazole represented by the following structural formula (I):

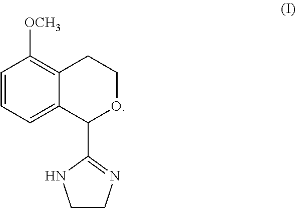 [SMILES: COc1cccc2c1CCOC2C1=NCCN1]

Tasipimidine and derivatives thereof have been disclosed in WO 2013/150173.

Tasipimidine exhibits agonistic activities on adrenergic alpha2 receptors, especially on alpha2A receptor, and can thus be used in the treatment of a disorder, condition or disease where an alpha2A agonist is indicated to be useful, for example, for use as a sedative or analgesic agent, and for use in the treatment of anxiety.

Besides therapeutic efficacy, the drug developer endeavors to provide a suitable form of the therapeutic agent that has properties relating to processing, manufacturing, storage stability, and/or usefulness as a drug. Accordingly, the discovery of a form that possesses some or all of these desired properties is vital to drug development. Tasipimidine and sulfate salt thereof may be prepared using the method described, for example, in WO 2019/106238. However, WO 2019/106238, or WO 2013/150173 provides no information at all about possible crystal modifications of tasipimidine sulfate.

### SUMMARY OF THE INVENTION

It has now been found that 2-(5-methoxyisochroman-1-yl)-4,5-dihydro-1H-imidazole (I), can exist in sulfate salt forms which are suitable for use in the manufacture of stable pharmaceutical products. In particular, crystalline polymorphic forms of sulfate salt were found which are chemically and physically stable under various manufacture and storage conditions. In addition, an amorphous form was also identified.

The novel sulfate salt forms of the present disclosure can be reproducibly prepared by choice of specially selected process conditions, e.g., choice of solvent system, duration of crystallization, etc., and they also exhibit properties that make them appropriate for drug development formulation use.

Accordingly, the present disclosure relates to novel crystalline sulfate salt forms of 2-(5-methoxyisochroman-1-yl)-4,5-dihydro-1H-imidazole (I) (referred herein as form 1, form 2, form 3 and form 4) and an amorphous form, and to the novel preparation methods thereof.

The disclosure also relates to pharmaceutical composition comprising at least one novel sulfate salt form of 2-(5-methoxyisochroman-1-yl)-4,5-dihydro-1H-imidazole (I), and to use of the novel forms in the treatment of anxiety, and for use as a sedative or analgesic agent, and other diseases were alpha2A agonism is desired.

The foregoing as well as other feature and advantages of the present teachings will be more fully understood from the following description and claims.

### BRIEF DESCRIPTION OF THE DRAWINGS

FIG. 1 shows the X-ray powder diffraction (XRPD) pattern of crystalline form 1 of sulfate salt of compound (I).

FIG. 2 shows the X-ray powder diffraction (XRPD) pattern of crystalline form 2 of sulfate salt of compound (I).

FIG. 3 shows the X-ray powder diffraction (XRPD) pattern of crystalline form 1+form 3 of sulfate salt of compound (I).

FIG. 4 shows the X-ray powder diffraction (XRPD) pattern of crystalline form 4 of sulfate salt of compound (I).

FIG. 5 shows the X-ray powder diffraction (XRPD) pattern of the amorphous form of sulfate salt of compound (I).

FIG. 6 shows the differential scanning calorimetry (DSC) thermogram of the crystalline form 1 of sulfate salt of compound (I).

FIG. 7 shows the differential scanning calorimetry (DSC) thermogram of the crystalline form 2 of sulfate salt of compound (I).

### DETAILED DESCRIPTION OF THE INVENTION

The present disclosure relates to novel sulfate salt forms of 2-(5-methoxyisochroman-1-yl)-4,5-dihydro-1H-imidazole (I). In particular, the present disclosure relates to sulfate salts of compound (I) in crystalline form. In addition, the present disclosure relates to sulfate salt of compound (I) in an amorphous form.

Novel forms (form 1, form 2, form 3, form 4 and an amorphous form) of sulfate salt of compound (I) have been characterized by an X-ray powder diffraction (XRPD) studies.

Accordingly, in one embodiment, the present disclosure relates to crystalline form 1 of sulfate salt of compound (I) having an X-ray powder diffraction pattern comprising characteristic peaks at about 5.8, 11.5, 13.9, 17.2 and 18.9 degrees 2-theta.

In one embodiment, the present disclosure relates to crystalline form 1 of sulfate salt of compound (I) having an X-ray powder diffraction pattern comprising characteristic peaks at about 5.8, 11.5, 13.9, 15.7, 17.2, 18.9, 23.8, 26.0, 27.2 and 28.3 degrees 2-theta.

In one embodiment, the crystalline form 1 of sulfate salt of compound (I) is further characterized by an X-ray powder diffraction pattern as depicted in FIG. 1.

In one embodiment, the present disclosure relates to crystalline form 2 of sulfate salt of compound (I) having an X-ray powder diffraction pattern comprising characteristic peaks at about 4.7, 9.3, 13.9, 14.6 and 18.7 degrees 2-theta.

In one embodiment, the present disclosure relates to crystalline form 2 of sulfate salt of compound (I) having an X-ray powder diffraction pattern comprising characteristic peaks at about 4.7, 9.3, 11.5, 12.0, 13.9, 14.6, 18.7, 21.7, 22.0, 25.3 and 26.5 degrees 2-theta.

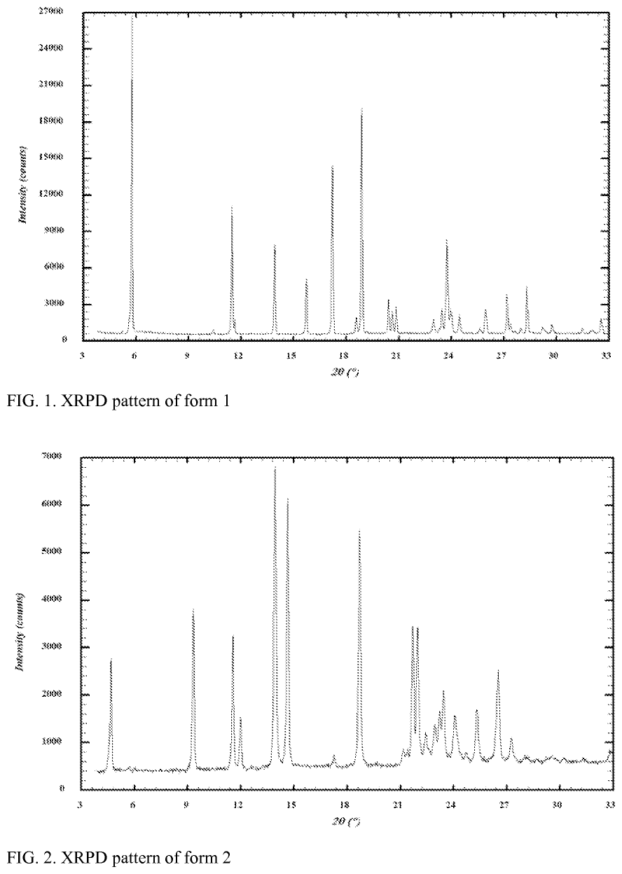

In one embodiment, the crystalline form 2 of sulfate salt of compound (I) is further characterized by an X-ray powder diffraction pattern as depicted in FIG. 2.

In one embodiment, the present disclosure relates to crystalline form 3 of sulfate salt of compound (I) having X-ray powder diffraction pattern comprising characteristic peaks at about 11.6, 17.7 and 23.6 degrees 2-theta.

In one embodiment, the present disclosure relates to a mixture of crystalline forms 1 and 3 of sulfate salt of compound (I) having X-ray powder diffraction pattern comprising characteristic peaks at about 5.8, 11.5, 11.6, 13.9, 17.2, 17.7, 18.9 and 23.6 degrees 2-theta.

In one embodiment, the mixture of crystalline forms 1 and 3 of sulfate salt of compound (I) is further characterized by an X-ray powder diffraction pattern as depicted in FIG. 3.

In one embodiment, the present disclosure relates to crystalline form 4 of sulfate salt of compound (I) having an X-ray powder diffraction pattern comprising characteristic peaks at about 5.8, 11.5, 15.5, 16.2, 17.0 and 17.2 degrees 2-theta.

In one embodiment, the present disclosure relates to crystalline form 4 of sulfate salt of compound (I) having an X-ray powder diffraction pattern comprising characteristic peaks at about 5.8, 8.5, 11.5, 15.5, 16.2, 17.0, 17.2, 18.8, 19.0, 24.1, 25.2, 25.6 and 32.5 degrees 2-theta.

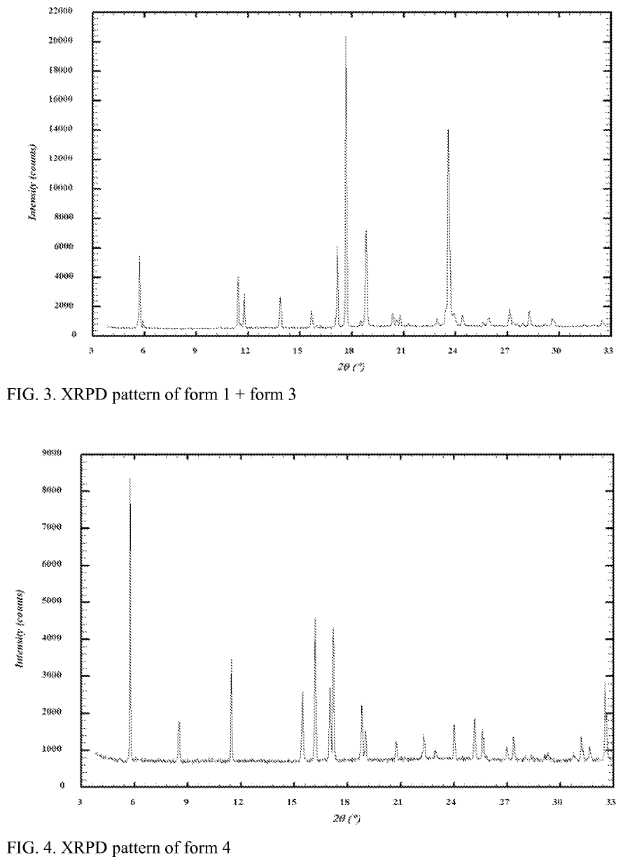

In one embodiment, the crystalline form 4 of sulfate salt of compound (I) is further characterized by an X-ray powder diffraction pattern as depicted in FIG. 4.

In one embodiment, the present disclosure relates to amorphous form of sulfate salt of compound (I).

In one embodiment, the amorphous form of sulfate salt of compound (I) is further characterized in that its X-ray powder diffraction pattern lacks discernible or sharp peaks and being substantially same as depicted in FIG. 5.

The above XRPD peak positions refer to values, when measured using CuKα radiation (λ=1.5418 Å). It is recognized by the skilled person that the X-ray powder diffraction pattern peak positions referred to herein can be subject to variations of ±0.2 degrees 2-theta according to various factors such as temperature, sample handling and instrumentation used.

The sulfate salt of compound (I) can be prepared as described, for example, in WO 2019/106238.

The crystalline form 1 of sulfate salt of compound (I) can be prepared, for example, by i) dissolving 2-(5-methoxyisochroman-1-yl)-4,5-dihydro-1H-imidazole sulfate in a suitable solvent at room or elevated temperature, ii) cooling the mixture, and iii) isolating the crystalline product. Suitable solvents include, for example, a mixture of acetone and water, a mixture of acetonitrile and water, a mixture of tetrahydrofuran (THF) and water, a mixture of methanol and ethyl acetate, a mixture of methanol and acetone, a mixture of methanol and heptane, a mixture of methanol, acetone and methyl acetate, a mixture of methanol and tert-butyl methyl ether (TBME), and a mixture of methanol and dichloromethane (DCM); such as the mixture of acetone and water. In particular, the crystalline form 1 can be prepared by dissolving 2-(5-methoxyisochroman-1-yl)-4,5-dihydro-1H-imidazole sulfate in a mixture of acetone and water at elevated temperature, for example at 50-70° C., preferably at 55-65° C. The ratio of acetone to water is suitably about 30:70 vol %. The solution is allowed to cool to about room temperature over one to several hours, for example about 2 hours. The mixture can be further cooled to about 5° C. followed by aging at about 5° C. for several hours, for example 72 hours. The precipitated crystalline form 1 can then be isolated, for example, by filtration or decantation, followed by slow solvent evaporation at about room temperature.

Alternatively, crystalline form 1 can be prepared, for example, by dissolving 2-(5-methoxyisochroman-1-yl)-4,5-dihydro-1H-imidazole sulfate in a suitable solvent at about room temperature followed by an anti-solvent addition. Suitable solvents include, for example, a mixture of ethanol and water, and methanol; such as the mixture of ethanol and water. The ratio of ethanol to water is suitably about 96:4 vol %. Suitable anti-solvents include, for example, TBME, heptane, acetone, and isopropyl acetate; such as TBME. Anti-solvent can be added to the solution, for example in 4 steps, at about room temperature under constant stirring. The suitable stirring time between the additions is, for example, about 5 minutes. After the anti-solvent addition the mixture is cooled to about 5° C. Or alternatively, anti-solvent can be pre-cooled at about 5° C. before mixing with the solution. After the anti-solvent addition, the mixture is aging at about 5° C. for several hours, for example about 72 hours. The precipitated crystalline form 1 can then be isolated, for example, by filtration or decantation, followed by slow solvent evaporation at about room temperature.

The crystalline form 2 of sulfate salt of compound (I) can be prepared, for example, by i) dissolving 2-(5-methoxyisochroman-1-yl)-4,5-dihydro-1H-imidazole sulfate in a suitable solvent at about room temperature, ii) heating the mixture iii) cooling the mixture, and iv) isolating the crystalline product. Suitable solvents include, for example, water, a mixture of ethanol and water, and a mixture of methanol and water; such as water. In particular, the crystalline form 2 can be prepared by dissolving 2-(5-methoxyisochroman-1-yl)-4,5-dihydro-1H-imidazole sulfate in water at about room temperature, followed by stirring at elevated temperature, for example, at 50-70° C., preferably at 55-65° C. The solution is allowed to cool to about room temperature over several hours, for example, about 2 hours. The mixture can be further cooled to about 5° C. followed by aging at about 5° C. for several hours, for example, about 72 hours. The crystalline form 2 can then be isolated, for example, by filtration or decantation, followed by slow solvent evaporation at about room temperature.

Alternatively, crystalline form 2 can be prepared, for example, by dissolving 2-(5-methoxyisochroman-1-yl)-4,5-dihydro-1H-imidazole sulfate in a suitable solvent at about room temperature followed by an anti-solvent addition. Suitable solvents include, for example, 2,2,2-trifluoroethanol (TFE), and water; such as TFE. Suitable anti-solvents include, for example, TBME, acetone, acetonitrile, 2-propanol, and THF; such as TBME. Anti-solvent can be pre-cooled at about 5° C. before mixing with the solution. After the anti-solvent addition, the mixture is suitably aging at about 5° C. for several hours, for example 72 hours. The precipitated crystalline form 2 can then be isolated, for example, by centrifugation, decantation and drying at room temperature.

The crystalline form 3 of sulfate salt of compound (I) can be prepared, for example, by stirring the slurry containing 2-(5-methoxyisochroman-1-yl)-4,5-dihydro-1H-imidazole sulfate and a mixture of THF and water. The ratio of THF to water is suitably about 90:10 vol %. The slurry is suitably stirred at about room temperature for a period sufficient to convert the starting material to its crystalline form 3, for example, about one week. The occurrence of crystalline form 3 of sulfate salt of compound (I) can be confirmed by XRPD analysis.

The crystalline form 4 of sulfate salt of compound (I) can be prepared, for example, by stirring the slurry containing 2-(5-methoxyisochroman-1-yl)-4,5-dihydro-1H-imidazole sulfate and 2-ethoxy ethanol. The slurry is suitably stirred at room temperature or at elevated temperature, for example at about at elevated temperature, for example, 30-50° C., preferably at 35-45° C., for a period sufficient to convert the starting material to its crystalline form 4, for example, about one week. The occurrence of crystalline form 4 of sulfate salt of compound (I) can be confirmed by XRPD analysis.

The amorphous form of sulfate salt of compound (I) can be suitably prepared by dissolving 2-(5-methoxyisochroman-1-yl)-4,5-dihydro-1H-imidazole sulfate in TFE and subsequently freezing the resulting clear solution with liquid nitrogen and then lyophilizing it for, for example, about 2 days. The sulfate salt of compound (I) can thus be obtained in amorphous form.

The crystalline forms of sulfate salt of compound (I) can be formulated into pharmaceutical dosage forms such as tablets, capsules, powders, suspensions or solutions together with excipients which are known in the art.

In one embodiment the present disclosure relates to a pharmaceutical composition comprising at least one of the crystalline forms 1, 2, 3, 4, or an amorphous form, preferably the crystalline form 1 of the sulfate salt of compound (I) and a pharmaceutically acceptable carrier or excipient. In one embodiment, the present disclosure relates to such compositions for use in the treatment or prevention of a disorder, condition or disease where an alpha2A agonist is indicated to be useful, for example, anxiety or condition wherein sedation or analgesia is needed.

In one embodiment, the present disclosure relates to the use of at least one of the crystalline forms 1, 2, 3, 4, or an amorphous form, preferably the crystalline form 1, of the sulfate salt of compound (I) as an active ingredient in the manufacture of a medicament for the treatment or prevention of a disorder, condition or disease where an alpha2A agonist is indicated to be useful, for example, anxiety or condition wherein sedation or analgesia is needed.

In one embodiment, the present disclosure relates to a method for the treatment of a disorder, condition or disease where an alpha2A agonist is indicated to be useful, for example, anxiety or condition wherein sedation or analgesia is needed, which method comprises administering to a mammal in need of such treatment a therapeutically effective amount of at least one of the crystalline forms 1, 2, 3, 4, or an amorphous form, preferably the crystalline form 1, of the sulfate salt of compound (I).

The present disclosure will be explained in more detailed by the following examples. The examples are meant for illustrating purposes only and do not limit the scope of the invention defined in the claims.

The polymorphic forms of compound (I) are characterized using the following procedures.

XRPD measurements were performed with the X-ray powder diffractometer Bruker D8 Advance at room temperature, using a copper filled X-ray tube (40 kV×40 mA) as the X-ray source, CuKα (2=1.5418 Å), a 0.6 mm divergence slit, 2.5° Soller slits on both the primary and secondary beams and the 1-dimensional LynxEye detector (with aperture angle of) 2.91593°. Data collection was done in the range of 3.8-33° 20 with 0.02° increments, at a scan speed of 0.3°/s.

Differential scanning calorimetry (DSC) measurements were performed with a Shimadzu DSC-60 calorimeter in the range of 20-300° C. with a heating rate of 10° C./min in crimped aluminum sample cell with nitrogen flow of 60 ml/min.

The single crystal measurements were performed on a Nonius Kappa-CCD diffractometer. Data were collected at 296 K. Data reduction was performed using HKL Scalepack and cell parameters were obtained using Denzo and Scalepak within θ range 1 to 32.5°. The structure was solved using direct methods by SHELXT-2014/7. The structure was refined by least square full matrix refinement using SHELXL-2014/7. All of the H atoms were found in the Fourier difference map and refined isotropically.

### Example 1: Preparation of 2-(5-methoxyisochroman-1-yl)-4,5-dihydro-1H-imidazole sulfate crystalline form 1

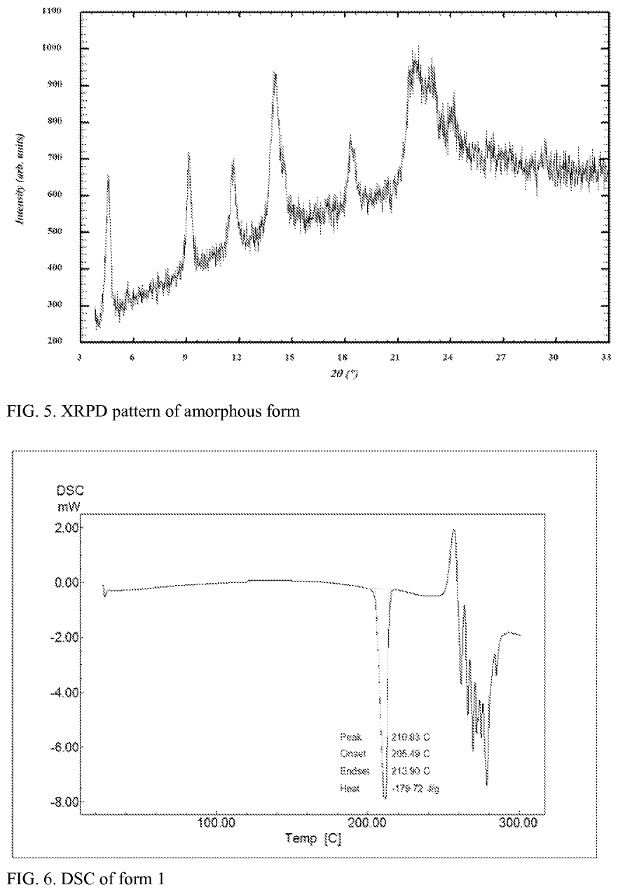

15 mg of 2-(5-methoxyisochroman-1-yl)-4,5-dihydro-1H-imidazole sulfate was dispensed in 200 μl of mixture of acetone and water (30:70 vol %) at room temperature (22-25° C.). The solution was stirred (600-1000 rpm) at room temperature followed by heating at 60° C. for 30 min. Next, the solution was cooled at room temperature during 2 h followed by ageing at 5° C. for 72 h. Finally, the solid was obtained by slow solvent evaporation at room temperature, in open vial for 3 days. The product was analyzed by XRPD and was found to be a crystalline form 1 (table 1). The X-ray powder diffraction pattern of form 1 is depicted in FIG. 1 and the differential scanning calorimetry (DSC) thermogram in FIG. 6. DSC analysis shows an endotherm with an onset temperature approximately 205° C.

TABLE 1
X-ray powder reflections (up to 33° 2θ) and intensities
(normalized) of crystalline form 1. The value 2θ [°]
represents the diffraction angle in degrees and the value d
[Å] represents the specified distances in Å between the lattice planes.

| 2θ [°] | d [Å] | I/Iₒ [%] |
|---|---|---|
| 5.77 | 15.31 | 100 |
| 10.41 | 8.49 | 1 |
| 11.47 | 7.71 | 39 |
| 11.63 | 7.60 | 4 |
| 13.91 | 6.36 | 27 |
| 15.73 | 5.63 | 17 |
| 17.20 | 5.15 | 51 |
| 18.58 | 4.77 | 5 |
| 18.88 | 4.70 | 69 |
| 20.40 | 4.35 | 10 |
| 20.65 | 4.30 | 7 |
| 20.86 | 4.25 | 8 |
| 22.99 | 3.87 | 4 |
| 23.47 | 3.79 | 7 |
| 23.75 | 3.74 | 29 |
| 23.97 | 3.71 | 7 |
| 24.46 | 3.64 | 5 |
| 25.64 | 3.47 | 2 |
| 25.97 | 3.43 | 7 |
| 27.19 | 3.28 | 11 |
| 27.38 | 3.26 | 3 |
| 27.59 | 3.23 | 1 |
| 27.97 | 3.19 | 1 |
| 28.32 | 3.15 | 14 |
| 29.21 | 3.06 | 2 |
| 29.75 | 3.00 | 3 |
| 31.44 | 2.84 | 1 |
| 32.06 | 2.79 | 1 |
| 32.56 | 2.75 | 4 |

### Example 2: Preparation of 2-(5-methoxyisochroman-1-yl)-4,5-dihydro-1H-imidazole sulfate crystalline form 2

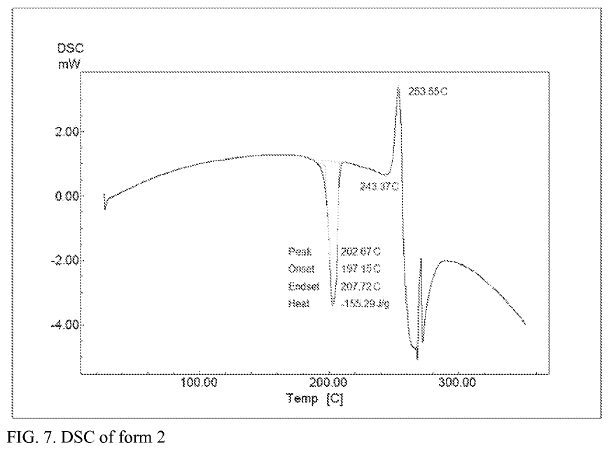

15 mg of 2-(5-methoxyisochroman-1-yl)-4,5-dihydro-1H-imidazole sulfate was dispensed in 200 μl of water at room temperature (22-25° C.). The solution was stirred (600-1000 rpm) at room temperature followed by heating at 60° C. for 30 min. Next, the solution was cooled at room temperature during 2 h followed by ageing at 5° C. for 72 h. Finally, the solid was obtained by slow solvent evaporation at room temperature, in open vial for 3 days. The product was analyzed by XRPD and was found to be a crystalline form 2 (table 2). The X-ray powder diffraction pattern of form 2 is depicted in FIG. 2 and the differential scanning calorimetry (DSC) thermogram in FIG. 7. DSC analysis shows an endotherm with an onset temperature approximately 197° C.

TABLE 2
X-ray powder reflections (up to 33° 2θ) and intensities
(normalized) of crystalline form 2. The value 2θ [°]
represents the difrraction angle in degrees and the value d
[Å] represents the specified distances in Å between the lattice planes.

| 2θ [°] | d [Å] | I/Iₒ [%] |
|---|---|---|
| 4.67 | 18.91 | 37 |
| 9.31 | 9.49 | 54 |
| 11.54 | 7.66 | 44 |
| 11.99 | 7.38 | 17 |
| 13.93 | 6.35 | 100 |
| 14.64 | 6.05 | 89 |
| 17.27 | 5.13 | 4 |
| 18.70 | 4.74 | 80 |
| 21.16 | 4.19 | 5 |
| 21.39 | 4.15 | 4 |
| 21.70 | 4.09 | 45 |
| 21.96 | 4.04 | 45 |
| 22.42 | 3.96 | 8 |
| 22.94 | 3.87 | 9 |
| 23.21 | 3.83 | 15 |
| 23.40 | 3.80 | 22 |
| 24.07 | 3.69 | 14 |
| 24.71 | 3.60 | 2 |
| 25.29 | 3.52 | 17 |
| 26.51 | 3.36 | 30 |
| 27.23 | 3.27 | 8 |

### Example 3: Preparation of 2-(5-methoxyisochroman-1-yl)-4,5-dihydro-1H-imidazole sulfate crystalline form 3

15 mg of 2-(5-methoxyisochroman-1-yl)-4,5-dihydro-1H-imidazole sulfate was dispensed in 200 μl of mixture of THF and water (90:10 vol %) at room temperature (22-25° C.) under magnetic stirring (600-1000 rpm). The resulting suspension was aged for 1 week at room temperature under magnetic stirring. After completing the ageing time, the solid was harvested and the wet powder was analyzed by XRPD, which indicates the occurrence of form 3 in mixture with form 1 (table 3). The X-ray powder diffraction pattern of form 1+form 3 is depicted in FIG. 3.

TABLE 3
X-ray powder reflections (up to 33° 2θ) and intensities
(normalized) of crystalline form 1 + 3. The value 2θ
[°] represents the diffraction andle in degrees
and the value d [Å] represents the specified distances
in Å between the lattice planes.

| 2θ [°] | d [Å] | I/Iₒ [%] |
|---|---|---|
| 5.71 | 15.46 | 25 |
| 5.91 | 14.94 | 3 |
| 11.41 | 7.75 | 18 |
| 11.59 | 7.63 | 2 |
| 13.86 | 6.39 | 11 |
| 15.67 | 5.65 | 6 |
| 15.92 | 5.56 | 1 |
| 16.21 | 5.46 | 1 |
| 17.14 | 5.17 | 28 |
| 17.67 | 5.02 | 100 |
| 18.54 | 4.78 | 2 |
| 18.84 | 4.71 | 34 |
| 20.33 | 4.36 | 4 |
| 20.60 | 4.31 | 2 |
| 20.80 | 4.27 | 3 |
| 22.91 | 3.88 | 2 |
| 23.40 | 3.80 | 5 |
| 23.60 | 3.77 | 61 |
| 23.92 | 3.72 | 4 |
| 24.39 | 3.65 | 3 |
| 25.61 | 3.48 | 1 |
| 25.96 | 3.43 | 3 |
| 27.14 | 3.28 | 6 |
| 27.38 | 3.25 | 2 |
| 27.90 | 3.20 | 1 |
| 28.27 | 3.15 | 5 |
| 29.62 | 3.01 | 2 |
| 32.49 | 2.75 | 2 |

### Example 4: Preparation of 2-(5-methoxyisochroman-1-yl)-4,5-dihydro-1H-imidazole sulfate crystalline form 4

15 mg of 2-(5-methoxyisochroman-1-yl)-4,5-dihydro-1H-imidazole sulfate was dispensed in 200 μl of 2-ethoxy ethanol at room temperature (22-25° C.) under magnetic stirring (600-1000 rpm). The resulting suspension was aged for 1 week at room temperature and at 40° C. under magnetic stirring. After completing the ageing time, the solid was harvested and the wet powder was analyzed by XRPD, which indicates the occurrence of form 4 (table 4). The X-ray powder diffraction pattern of form 4 is depicted in FIG. 4.

TABLE 4
X-ray powder reflections (up to 33° 2θ) and intensities
(normalized) of crystalline form 4. The value 2θ [°]
represents the diffraction angle in degrees and the value d
[Å] represents the specified distances in Å between the lattice planes.

| 2θ [°] | d [Å] | I/Iₒ [%] |
|---|---|---|
| 5.75 | 15.36 | 100 |
| 8.51 | 10.38 | 15 |
| 11.47 | 7.71 | 36 |
| 15.47 | 5.72 | 24 |
| 16.19 | 5.47 | 50 |
| 17.02 | 5.21 | 25 |
| 17.20 | 5.15 | 46 |
| 18.81 | 4.71 | 20 |
| 19.03 | 4.66 | 10 |
| 20.75 | 4.28 | 6 |
| 22.30 | 3.98 | 9 |
| 22.96 | 3.87 | 3 |
| 24.01 | 3.70 | 13 |
| 25.16 | 3.54 | 14 |
| 25.61 | 3.48 | 11 |
| 26.99 | 3.30 | 4 |
| 27.37 | 3.26 | 8 |
| 28.37 | 3.14 | 3 |
| 29.13 | 3.06 | 3 |
| 29.31 | 3.05 | 3 |
| 30.77 | 2.90 | 3 |
| 31.19 | 2.86 | 9 |
| 31.65 | 2.82 | 5 |
| 32.54 | 2.75 | 28 |

### Example 5: Preparation of 2-(5-methoxyisochroman-1-yl)-4,5-dihydro-1H-imidazole sulfate in amorphous form

15 mg of 2-(5-methoxyisochroman-1-yl)-4,5-dihydro-1H-imidazole sulfate was mixed with 1 ml of 2,2,2-trifluoroethanol (TFE) at room temperature. The mixture was stirred for 2-3 minutes, and the resulting clear solution was frozen with liquid nitrogen and then lyophilized for about 2 days. The freeze-drying conditions were 1) main drying: 0.20 mbar, −36° C. for about 47 hours, 2) final drying: 6 mbar, 0° C. for 1 hour. The resulting white powder was harvested and analyzed by XRPD, which indicates the occurrence of an amorphous form. The X-ray powder diffraction pattern of an amorphous form is depicted in FIG. 5.

### EXPERIMENT 1: Slurry Experiments Started with 2-(5-methoxyisochroman-1-yl)-4,5-dihydro-1H-imidazole sulfate crystalline form 1

The slurry experiments (SLC, SL and SLT) were carried out with the crystalline form 1 in different conditions. 15 mg of starting material were mixed with 200 μl of solvent or solvent mixture (at a concentration of about 75 mg/ml). The mixture was homogenized by vortexing for 10 seconds. The resulting suspensions were aged at different conditions: a) at 5° C. under magnetic stirring (SLC), b) at room temperature under magnetic stirring (SL), and c) at 40° C. in the shaker at 700 rpm (SLT). During the aging time, the samples were checked regularly, and solvent was added if needed. After completing the aging time, the vials were opened, and the (wet) powders were analyzed by XRPD. The results are shown in Table 6.

TABLE 6
Experimental details and results of
the SL, SLC and SLT experiments.

| | Water | Resulting crystalline form | | |
|---|---|---|---|---|
| Solvent system | activity | SLC | SL | SLT |
| Water | 1 | Form 2 | Form 2 | Form 2 |
| Ethanol | — | Form 1 | Form 1 | Form 1 |
| 2-Methyltetrahydrofuran | — | Form 1 | Form 1 | Form 1 |
| 2-Butanone | — | Form 1 | Form 1 | Form 1 |
| 2-Ethoxy ethanol | — | Form 1 + | Form 4 | Form 4 |
| | | Form 4 | | |
| Methanol/Water | >0.9 | Form 2 | Form 2 | Form 2 |
| (20:80 vol %) | | | | |
| Ethanol/Water | >0.9 | Form 2 | Form 2 | Form 2 |
| (26:74 vol %) | | (+Form 1) | | |
| Acetonitrile/Water | 0.60 | Form 1 | Form 1 | Form 1 |
| (96:4 vol %) | | | | |
| Acetone/Water | 0.75 | Form 1 | Form 1 | Form 1 |
| (86:14 vol %) | | | | |
| THF/Water | 0.88 | Form 1 | Form 1 + | Form 1 |
| (90:10 vol %) | | | Form 3 | |

Most experiments indicated the occurrence of form 1, form 2 and mixtures thereof. The crystalline form 2 is favored under higher water activity conditions (about 0.9). In addition, form 4 and form 3 were detected in the wet solids obtained from 2-ethoxy ethanol and THF/water, respectively.

### EXPERIMENT 2: Single Crystal X-Ray Diffraction Data of 2-(5-methoxyisochroman-1-yl)-4,5-dihydro-1H-imidazole sulfate crystalline form 1

Unit cell parameters of crystalline form 1 of sulfate salt of compound (I) were determined from single crystal X-ray diffraction data and are summarized below, T=296 (2) K, radiation wavelength MoKα (λ=0.71073 Å), crystal size 0.3×0.15×0.06 mm³, empirical formula [(C₁₃H₁₇N₂O₂) (HSO₄)].

| | |
|---|---|
| Crystal system | Triclinic |
| Space group | P-1 |

| | | |
|---|---|---|
| Unit cell dimensions | a = 4.9249(3) Å | α = 95.221(3)° |
| | b = 9.7884(6) Å | β = 96.846(3)° |
| | c = 15.7497(12) Å | γ = 103.832(3)° |
| Volume | V = 726.33(8) Å³ | |

| | |
|---|---|
| Z | 2 |
| Goodness-of-fit on F² | 1.052 |

### EXPERIMENT 3: Short-Term Accelerated Stability Test

The two crystalline forms (form 1 and form 2) were openly exposed for 1 week at 40° C., 75% RH to assess their physical stability on storage at elevated temperature and relative humidity. After exposure the solids were re-analyzed by XRPD. The results are shown in Table 7.

TABLE 7
Short-term accelerated stability of forms 1 and 2.

| Starting | Physical stability at |
|---|---|
| material | accelerated conditions |
| Form 1 | Stable |
| Form 2 | Partially transforms into form 1 |

The results shows that crystalline form 1 shows good stability while form 2 partially changes to form 1. In addition, forms 3 and 4 were found unstable already at room temperature and exhibiting conversion into form 1.

A person skilled in the art will appreciate that the embodiments described herein can be modified without departing from the inventive concept. A person skilled in the art also understands that the present disclosure is not limited to the particular embodiments disclosed but is intended to also cover modifications of the embodiments that are within the scope of the present disclosure.

1. A compound which is a crystalline form 1 of the sulfate salt of 2-(5-methoxyisochroman-1-yl)-4,5-dihydro-1H-imidazole (I) characterized by an X-ray powder diffraction pattern comprising peaks at 5.8±0.2, 11.5±0.2, 13.9±0.2, 17.2±0.2 and 18.9±0.2 degrees 2-theta.

2. The compound according to claim 1, having substantially the same X-ray diffraction pattern as shown in FIG. 1.

3. A compound which is a crystalline form 2 of the sulfate salt of 2-(5-methoxyisochroman-1-yl)-4,5-dihydro-1H-imidazole (I) characterized by an X-ray powder diffraction pattern comprising peaks at 4.7±0.2, 9.3±0.2, 13.9±0.2, 14.6±0.2 and 18.7±0.2 degrees 2-theta.

4. The compound according to claim 3, having substantially the same X-ray diffraction pattern as shown in FIG. 2.

5. A compound which is a crystalline form 3 of the sulfate salt of 2-(5-methoxyisochroman-1-yl)-4,5-dihydro-1H-imidazole (I) characterized by an X-ray powder diffraction pattern comprising peaks at 11.8±0.2, 17.7±0.2 and 23.6±0.2 degrees 2-theta.

6. A mixture comprising the compound of claim 5 and a compound which is a crystalline form 1 of the sulfate salt of 2-(5-methoxyisochroman-1-yl)-4,5-dihydro-1H-imidazole (I) characterized by an X-ray powder diffraction pattern comprising peaks at 5.8±0.2, 11.5±0.2, 13.9±0.2, 17.2±0.2 and 18.9±0.2 degrees 2-theta.

7. The mixture according to claim 6, having substantially the same X-ray diffraction pattern as shown in FIG. 3.

8. A compound which is a crystalline form 4 of the sulfate salt of 2-(5-methoxyisochroman-1-yl)-4,5-dihydro-1H-imidazole (I) characterized by an X-ray powder diffraction pattern comprising peaks at 5.8±0.2, 11.5±0.2, 15.5±0.2, 16.2±0.2, 17.0±0.2 and 17.2±0.2 degrees 2-theta.

9. The compound according to claim 8, having substantially the same X-ray diffraction pattern as shown in FIG. 4.

10. A compound which is an amorphous form of the sulfate salt of 2-(5-methoxyisochroman-1-yl)-4,5-dihydro-1H-imidazole (I).

11. The compound according to claim 10, having substantially the same X-ray diffraction pattern as shown in FIG. 5.

12. A method of preparing the compound according to claim 1, comprising dissolving the sulfate salt of 2-(5-methoxyisochroman-1-yl)-4,5-dihydro-1H-imidazole (I) in a mixture of acetone and water, cooling the mixture, and isolating the crystalline product.

13. A method of preparing the compound according to claim 3, comprising dissolving the sulfate salt of 2-(5-methoxyisochroman-1-yl)-4,5-dihydro-1H-imidazole (I) in water, cooling the mixture, and isolating the crystalline product.

14. A method of preparing the compound according to claim 5, comprising preparing a slurry of the sulfate salt of 2-(5-methoxyisochroman-1-yl)-4,5-dihydro-1H-imidazole (I) in a mixture of THF and water, and allowing for sufficient time to induce crystallization of the product.

15. A method of preparing the compound according to claim 8, comprising preparing a slurry of the sulfate salt of 2-(5-methoxyisochroman-1-yl)-4,5-dihydro-1H-imidazole (I) in 2-ethoxy ethanol, and allowing for sufficient time to induce crystallization of the product.

16. A method of preparing the compound according to claim 10, comprising dissolving the sulfate salt of 2-(5-methoxyisochroman-1-yl)-4,5-dihydro-1H-imidazole (I) in 2,2,2-trifluoroethanol, freezing the mixture, and removing the solvent by freeze-drying to obtain an amorphous product.

17. A pharmaceutical composition comprising the compound according to claim 1 as an active ingredient together with one or more excipients.

18. A method of treating a disorder, condition or disease where an alpha2A agonist is indicated to be useful, comprising administering the compound of claim 1 to a subject in need thereof.

19. The method of claim 18, wherein the disorder is anxiety.

20. A pharmaceutical composition comprising the compound according to claim 3 as an active ingredient together with one or more excipients.

21. A method of treating a disorder, condition or disease where an alpha2A agonist is indicated to be useful, comprising administering the compound of claim 3 to a subject in need thereof.

22. The method of claim 21, wherein the disorder is anxiety.

23. A pharmaceutical composition comprising the compound according to claim 5 as an active ingredient together with one or more excipients.

24. A method of treating a disorder, condition or disease where an alpha2A agonist is indicated to be useful, comprising administering the compound of claim 5 to a subject in need thereof.

25. The method of claim 24, wherein the disorder is anxiety.

26. A pharmaceutical composition comprising the compound according to claim 8 as an active ingredient together with one or more excipients.

27. A method of treating a disorder, condition or disease where an alpha2A agonist is indicated to be useful, comprising administering the compound of claim 8 to a subject in need thereof.

28. The method of claim 27, wherein the disorder is anxiety.

29. A pharmaceutical composition comprising the compound according to claim 10 as an active ingredient together with one or more excipients.

30. A method of treating a disorder, condition or disease where an alpha2A agonist is indicated to be useful, comprising administering the compound of claim 10 to a subject in need thereof.

31. The method of claim 30, wherein the disorder is anxiety.
